SEQUENCE LISTING (15 sequences)

SEQ ID NO: 1 - DNA, 34 bp, Artificial Sequence
  Description of Artificial Sequence Synthetic oligonucleotide
gttagaggggcatgtccgggcatgtccgggcaga

SEQ ID NO: 2 - DNA, 34 bp, Artificial Sequence
  Description of Artificial Sequence Synthetic oligonucleotide
tctgcccggacatgcccggacatgcccctctaac

SEQ ID NO: 3 - DNA, 30 bp, Artificial Sequence
  Description of Artificial Sequence Synthetic oligonucleotide
atcatgaacatgtctaagcatgctgagctc

SEQ ID NO: 4 - DNA, 30 bp, Artificial Sequence
  Description of Artificial Sequence Synthetic oligonucleotide
tagtacttgtacagattcgtacgactcgag

SEQ ID NO: 5 - DNA, 30 bp, Artificial Sequence
  Description of Artificial Sequence Synthetic oligonucleotide
tcattcacaagttagagacaagcctagctc

SEQ ID NO: 6 - DNA, 30 bp, Artificial Sequence
  Description of Artificial Sequence Synthetic oligonucleotide
agtaagtgttcaatctctgttcggatcgag

SEQ ID NO: 7 - DNA, 30 bp, Artificial Sequence
  Description of Artificial Sequence Synthetic oligonucleotide
acctggggaatttccgggaatttccgctga

SEQ ID NO: 8 - DNA, 30 bp, Artificial Sequence
  Description of Artificial Sequence Synthetic oligonucleotide
tggaccccttaaaggcccttaaaggcgact

SEQ ID NO: 9 - DNA, 30 bp, Artificial Sequence
  Description of Artificial Sequence Synthetic oligonucleotide
atcatgaaaggtggatttaggtggaagctc

SEQ ID NO: 10 - DNA, 30 bp, Artificial Sequence
  Description of Artificial Sequence Synthetic oligonucleotide
tagtactttccacctaaatccaccttcgag

SEQ ID NO: 11 - DNA, 34 bp, Artificial Sequence
  Description of Artificial Sequence Synthetic oligonucleotide
gttagagatgcgagagttcagtaagcggggcaga

SEQ ID NO: 12 - DNA, 34 bp, Artificial Sequence
  Description of Artificial Sequence Synthetic oligonucleotide
tctgccccgcttactgaactctcgcatctctaac

SEQ ID NO: 13 - DNA, 13 bp, Artificial Sequence
  Description of Artificial Sequence Synthetic oligonucleotide
  modified_base (6)..(8): a, c, t, g, unknown or other
ggtcannntgacc

SEQ ID NO: 14 - DNA, 20 bp, Artificial Sequence
  Description of Artificial Sequence Synthetic oligonucleotide
ggacaagtcaggacttgcag

SEQ ID NO: 15 - DNA, 13 bp, Artificial Sequence
  Description of Artificial Sequence Synthetic oligonucleotide
ggtcactgtgacc

(19) United States
(12) Patent Application Publication
(10) Pub. No.: US 2018/0172678 A1
(43) Pub. Date: Jun. 21, 2018

(54) Dynamic Light Scattering Nanoplatform For High-Throughput Drug Screening

(71) Applicants: Agency for Science, Technology and Research, Singapore (SG); National University of Singapore, Singapore (SG)

(72) Inventors: Yen Nee TAN, Singapore (SG); Xinting ZHENG, Singapore (SG); Farid GHADESSY, Singapore (SG); Walter Leng Peng GOH, Singapore (SG); David LANE, Singapore (SG); Lin-Yue Lanry YUNG, Singapore (SG)

(21) Appl. No.: 15/382,570

(22) Filed: Dec. 16, 2016

(51) Int. Cl. G01N 33/53; G01N 33/574

(52) U.S. Cl. CPC G01N 33/5308; G01N 2500/02; G01N 2500/20; G01N 33/574

(57) ABSTRACT

Methods, systems and nanoprobes for identifying sequence-specific transcription factor DNA interactions for drug and/or disease screening are provided. The method includes homogeneously mixing plasmonic metal nanoparticle probes with protein samples as an assay in multi-well plates. The plasmonic metal nanoparticle probes comprise a plurality of plasmonic metal nanoparticles and a specific DNA response element and the protein samples bind with the specific response elements to form an assembly of the plasmonic metal nanoparticle probes. The method further includes measuring particle size distribution of the assembly of the plasmonic metal nanoparticle probes in the solution by dynamic light scattering and determining one or more sequence-specific transcription factor DNA interactions from a curve of the particle size distribution determined from light scattered by the dynamic light scattering. The nanoprobe includes a plurality of plasmonic metal nanoparticles and a DNA linker. The DNA linker forms a link between the two plasmonic metal nanoparticles, the DNA linker including a double stranded region encoding a specific response element.

### TECHNICAL FIELD

The present invention generally relates to methods and devices for transcription factor-DNA interaction determination, and more particularly relates to methods and devices for using dynamic light scattering for high-throughput sequence-specific transcription factor-DNA interaction determination for drug and/or disease screening.

### BACKGROUND

The investigation of important biomolecular events such as DNA mutation and gene transcription have been made possible with the advent of nanotechnology. Various nanosensing probes, such as metal nanoparticles, quantum dots, and silicon nanowires have been utilized to lend insight into the intertwining complexities between biomolecules, by transducing ‘invisible’ biological signals into measurable output. In particular, gold nanoparticles (AuNPs) have added a new dimension to the realm of biosensing through their exhibition of localized surface plasmon resonance (LSPR). The strong absorbance and scattering characteristics of AuNPs at the visible light region render them as ideal sensing probes for various bioassay developments based on different optical responses. For example, the interparticle-distance dependent plasmonic coupling of AuNPs has been utilized to design colorimetric assays for biomolecular detection. However, a high concentration of targets are needed in the colorimetric assay to aggregate the AuNP to elicit appreciable color changes to be visibly perceived, which results in less than ideal sensitivity. In addition, it is not suitable for use in coloured samples such as blood, which would interfere with the red to purple/blue transition in observations by the naked eye or using UV-vis spectroscopy.

Given that low sensitivity is one of the main limitations of gold nanoparticles-based colorimetric assays, methods such as biobarcode and silver staining amplifications have been carried out to address the problem, but such methods are still complicated and time consuming. As such, it has been shown that AuNP probes carrying recognition sequences for protein-protein binding and DNA-DNA hybridization would cluster and aggregate in the presence of their target binders. The increase in particle size led to a wholesale shift in the population distribution from tens of nm to the hundreds nm range. Given that larger AuNPs show greater scattering cross section, the overall size increase from the aggregation amplifies the readout, further enhancing the sensitivity and clarity of the readout.

If a sensitive platform for detecting AuNP-transduced biorecognition signals and biodiagnostic strategies involving increase in AuNP size could be addressed, such a platform could be used for cancer screening. Cancer is one of the prevalent causes of death worldwide and can take more than 200 diverse forms, including lung cancer, prostate cancer, breast cancer, cervical cancer, ovarian cancer, hematologic cancer, colon cancer, or leukemia. Environmental factors as well as genetic factors have been linked with an increased threat in the development and progression of cancer. However, many developed cancer therapies are specific only to a certain kind of cancer. Among cancer treatments, chemotherapy is a more ‘general’ anti-cancer method but is very invasive and non-targeting. Chemotherapy drugs kill both cancer cells and normal cells, thereby bringing severe side effects to patients.

The p53 protein is a general tumor suppressor which governs cell fates, thus it has been called “the guardian of the genome”. As a typical transcription factor, p53 binds to specific DNA response elements (REs) which regulate the expression of target genes. Approximately half of all cancers have been found to result from mutations in p53, thereby making p53 pathway a prime target for cancer therapy development. Of the dozens of p53 drugs currently in development, the vast majority simply try to boost levels of healthy p53. When p53 proteins are mutated, they lose their ability to bind to specific DNA promoter sequences containing DNA REs and, thus, are unable to trigger processes that safeguard a normal cell such as cell cycle arrest, DNA repair or apoptosis.

The discovery of a drug that is able to restore mutant p53's DNA binding ability is of high clinical importance and promises to change the landscape for cancer treatment and for treatment of other diseases that involve misfolded proteins such as Alzheimer's disease. More generally, if the tumor suppressor functions of p53 could be activated by an anticancer drug, it would greatly improve the drug efficacy. It is envisioned that a p53 activation or reactivation drug will present a general strategy to treat many kinds of cancer with just a few drugs. Unfortunately, there is a lack of a simple, fast, sensitive and high-throughput drug screening assay to target p53 activation in a complex biological setting. In addition, estrogen receptor (ER) is a protein biomarker that has significant implication in breast cancer prognosis and treatment. Thus, a sensitive and selective method for detection of binding interactions of ER with its consensus DNA containing estrogen response element (ERE) in a fast and simple manner is highly desirable.

Conventional methods that have been used to ascertain p53-DNA binding include gel shift assay, DNA footprinting, fluorescence anisotropy, Chromatin Immunoprecipitation (ChIP), Surface Plasmon Resonance (SPR) and enzyme-linked immunosorbent assay (ELISA). These methods are mostly heterogeneous-phase assays which involve multiple surface treatments, a high level of technical expertise, and require expensive reagents and sophisticated instrumentation. Thus, they are not suitable for a high-throughput drug screening targeting p53 pathway. In addition, these methods have high background noise and are mainly used to detect purified protein samples. More recently, two new approaches, multiplex in vitro binding assay and microsphere assay for protein-DNA binding (MAPD), have been designed to detect p53-DNA binding in in vitro transcription/translation (IVT) samples or nuclear extracts in a semi-quantitative manner. These assays still suffer from tedious procedures and, more critically, reliance on multiple expensive reagents such as antibodies, primers and beads for signal readout.

Dynamic light scattering (DLS) can also detect 100 nm AuNPs at as low as fM level without added processing or amplification, which makes DLS a sensitive platform for detecting AuNP-transduced biorecognition signals, and also biodiagnostic strategies involving increase in AuNP size. However, as in most aggregation-based systems, particle aggregation is an uncontrolled process, with the biomolecular targets causing the AuNPs to aggregate and grow extensively, leading to large variations in AuNP aggregate size and complex DLS readouts that are complicated to analyze especially for more subtle size changes. Greater control over the probe-analyte interaction process is necessary to leverage the size growth of AuNPs, detected by DLS machine.

Thus, what is urgently needed is a simple, fast, sensitive, label-free and high-throughput assay platform to identify and evaluate binding interactions directly in live cells, cell lysates and/or other biological protein samples. Furthermore, other desirable features and characteristics will become apparent from the subsequent detailed description and the appended claims, taken in conjunction with the accompanying drawings and this background of the disclosure.

### SUMMARY

According to at least one embodiment of the present invention, a method for identifying sequence-specific transcription factor DNA interactions is provided. The method includes homogeneously mixing plasmonic metal nanoparticle probes with protein samples as an assay in multi-well plates. The plasmonic metal nanoparticle probes comprise a plurality of plasmonic metal nanoparticles and a specific response element and the protein samples bind with the specific response elements to form an assembly of the plasmonic metal nanoparticle probes. The method further includes measuring particle size distribution of the assembly of the plasmonic metal nanoparticle probes in the solution by dynamic light scattering and determining one or more sequence-specific transcription factor DNA interactions from a curve of the particle size distribution determined from light scattered by the dynamic light scattering.

According to another embodiment of the present invention, a nanoprobe is provided. The nanoprobe includes a plurality of plasmonic metal nanoparticles and a DNA linker. The DNA linker forms a link between the two plasmonic metal nanoparticles, the DNA linker including a double stranded region encoding a specific response element.

According to a further embodiment of the present invention a system for drug screening is provided. A system includes an assay, multi-well plates for combining the assay with a biological sample, and a measurement device. The assay includes a plurality of specific transcription factor DNA response elements having large light scattering dimensions when binded to drug-activated or -reactivated transcription factor proteins. The biological sample includes drug-activated or -reactivated transcription factor proteins. And the measurement device determines binding affinity of the drug-activated or -reactivated transcription factor proteins with the plurality of specific transcription factor DNA response elements by measuring particle size dimensions in the multi-well plates by dynamic light scattering.

And according to yet a further embodiment of the present invention a system for disease screening is provided. The system includes an assay, multi-well plates and a measurement device. The assay includes a plurality of specific DNA response elements having large light scattering dimensions when binded to receptor elements. The assay is combined with a disease screening biological sample in the multi-well plates. And the measurement device determines binding of the specific DNA response elements with the receptor elements in the disease screening biological sample by measuring particle size dimensions in the multi-well plates by dynamic light scattering.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 depicts a schematic illustration of the drug screening assay principles for mutant p53 reactivation using specially designed dumbbell-shaped p53 reaction element (RE)-linked gold nanoprobes (AuNPs) as ultrasensitive sensing probes in dynamic light scattering (DLS) measurements in accordance with a present embodiment.

FIG. 2, comprising FIGS. 2A and 2B, depicts graphs of wildtype p53 protein bound PUMA-AuNP nano-dumbbell probes measured by DLS in accordance with the present embodiment, wherein FIG. 2A depicts a graph of size distribution wildtype p53 protein bound PUMA-AuNP nano-dumbbell probes measured by DLS and FIG. 2B depicts a calibration plot of wildtype p53 protein bound PUMA-AuNP nano-dumbbell probes measured by DLS.

FIG. 3, comprising FIGS. 3A and 3B, depicts graphs of the wildtype p53 and PUMA-AuNP binding event in accordance with the present embodiment, wherein FIG. 3A depicts a graph of real-time monitoring of the wildtype p53 and PUMA-AuNP binding event and FIG. 3B depicts a statistical plot showing size shift as it increases with incubation time.

FIG. 4, comprising FIGS. 4A and 4B, depicts graphs of a DNA sequence selectivity study of wildtype p53 binding in accordance with the present embodiment, wherein FIG. 4A depicts a graph of size distribution and FIG. 4B depicts a statistical bar graph of wildtype p53 binding to Scr-AuNPs linked by scrambled sequence versus wildtype p53 binding to PUMA-AuNP nano-dumbbell probes with p53-specific PUMA sequence in accordance with the present embodiment.

FIG. 5, comprising FIGS. 5A and 5B, depicts graphs of specific detection of wtp53 versus mutant p53 proteins onto PUMA-AuNPs in accordance with the present embodiment, wherein FIG. 5A depicts a bar graph showing that only wildtype p53 binds to PUMA-AuNP nano-dumbbell probes to cause size increases while non-binding mutant p53 (R273H), BSA and HSA do not interact with the PUMA-AuNPs and FIG. 5B depicts size distribution plots showing suppression of the signal from the non-specific BSA proteins in the presence of PUMA-AuNPs while the wildtype p53 binds to the PUMA-AuNP nano-dumbbell probes in accordance with the present embodiment.

FIG. 6 depicts an illustration summarizing drug screening applications in accordance with the present embodiment.

FIG. 7, comprising FIGS. 7A and 7B, depicts bar graphs showing cell lysate analysis statistics in accordance with the present embodiment, wherein FIG. 7A depicts wildtype p53 cell lysate and three mutant p53 cell lysates (G245S, R273H and R175H) and FIG. 7B depicts detection of wildtype p53 in PonA induced p53 cell lysates versus a p53 knockdown cell lysate (EI), and an anticancer drug (ActD, Dox, Etoposide)-treated cell lysate.

FIG. 8 depicts a bar graph of detection of a wildtype p53 expression level in drug treated HCT116 cell lysates in accordance with the present embodiment.

FIG. 9 depicts a schematic diagram showing the process of mutant reactivation drug screening in accordance with the present embodiment.

FIG. 10, comprising FIGS. 10A and 10B, depicts bar graphs of mutant reactivation drugs screening in accordance with the present embodiment, wherein FIG. 10A depicts a bar graph comparing binding of reactivation of mutant p53 (R175H) with PUMA-AuNP nano-dumbbell probes by COTI-2 and Prima-1^{met} at high concentrations (++) to NSC drug effects at low and high concentrations and FIG. 10B depicts a bar graph comparing binding of reactivation of mutant p53 (R273H) to COTI-2 and Prima-1^{met} at high concentrations (++) with the NSC exerting no drug effect due to its specificity to the R175H mutant.

FIG. 11, comprising FIGS. 11A and 11B, depicts schematics and bar graphs for evaluation of DNA sequence binding affinity in accordance with the present embodiment, wherein FIG. 11A depicts a schematic for competition assay for evaluation of the DNA sequence binding affinity and FIG. 11B depicts statistical analysis showing the DNA sequence binding affinity of excess free p53-binding ConA sequence, wildtype p53 to PUMA-AuNP nano-dumbbell probes, other promoter sequences such as GADD45 and Bax, a mutated ConA sequence and WRNC.

FIG. 12 depicts a schematic illustration and DLS readout in accordance with the present embodiment showing conjugation of ssDNA seq A and Seq B to AuNPs exhibiting a single peak under DLS, an ERE-containing AuNP dumbbell probes evidencing a single peak right-shifted as compared to the conjugates, and addition of ERβ to the ERE-containing AuNP dumbbell probes which presents a two-peak readout with an additional complex peak.

FIG. 13, comprising FIGS. 13A, 13B and 13C, depicts graphs showing DLS analysis of the ERβ interaction of FIG. 12 with various AuNP dumbbell probes in accordance with the present embodiment, wherein FIG. 13A depicts a graph of the DLS analysis of the ERβ interaction with unmodified citrate-anion capped AuNP dumbbell probes, FIG. 13B depicts a graph of the DLS analysis of the ERβ interaction with OEG passivated AuNP dumbbell probes, and FIG. 13C depicts a graph of the DLS analysis of the ERβ interaction with AuNP dumbbell probes bearing one strand of ssDNA.

FIG. 14, comprising FIGS. 14A and 14B, depicts DLS complex peak development of the ERE-containing AuNP dumbbell probe interaction with ERβ in accordance with the present embodiment, wherein FIG. 14A depicts graphs of a time-dependent study of the ERE-containing AuNP dumbbell probe interaction with 10 nM ERβ over thirty minutes and FIG. 14B depicts graphs of a concentration-dependent study of the ERE-containing AuNP dumbbell probe interaction with ERβ at thirty minutes after ERβ addition.

FIG. 15, comprising FIGS. 15A and 15B, depicts DLS graphs of different dumbbell probe interaction with the ERβ in accordance with the present embodiment, wherein FIG. 15A depicts a graph of AC dumbbell probes consisting of AuNPs joined by dsDNA containing mutated ERE sequence interacting with the ERβ and FIG. 15B depicts a graph of ERE-containing AuNP dumbbell probes queried with a non-specific protein BSA and interacting with the ERβ.

FIG. 16 depicts a graph of DLS readouts of ERE-containing AuNP trimer probes incubated with the ERβ in accordance with the present embodiment.

Skilled artisans will appreciate that elements in the figures are illustrated for simplicity and clarity and have not necessarily been depicted to scale.

### DETAILED DESCRIPTION OF CERTAIN EMBODIMENTS

The following detailed description is merely exemplary in nature and is not intended to limit the invention or the application and uses of the invention. Furthermore, there is no intention to be bound by any theory presented in the preceding background of the invention or the following detailed description. It is the intent of the present embodiment to present a unique DNA-assembled gold nanoparticle (AuNP) probe for dynamic light scattering (DLS) sensing of transcription factors. A specific response element sequence is incorporated into DNA linkers used to bridge the AuNPs in the AuNP probe. Coupled with the DLS measurement, this AuNP probe-based DLS detection system provides specific readouts in the presence of target molecule. This unique optical signature enables the nanostructures to be used in conjunction with a DLS platform to study transcription factor-DNA interactions. In addition, the AuNP nanoprobes could also suppress the light-scattering signal from unbound proteins and other interfering factors (e.g., buffer background), and provide highly sensitive detection of target proteins in complex biological samples such as cell lysates. Thus, the AuNP probe coupled with DLS measurement is a simple (mix and test), rapid (readout in ˜5 min) and sensitive (low nM levels) platform to detect sequence-specific protein-DNA binding event.

In addition, a new dynamic light scattering (DLS) based high-throughput anticancer drug screening nanoplatform targeting the p53 pathway. The nanoplatform in accordance with a present embodiment is capable of quantitatively measuring p53-DNA binding in real-time, determining whether a drug can reactivate mutant p53 protein to restore its sequence-specific binding ability to p53 reaction elements (REs) or increase wildtype p53 activity, and evaluating sequence specificity for drug validation. The nanoplatform capitalizes on the large scattering dimension of gold nanoprobes (AuNPs) (approximately 10⁶-fold larger than fluorescent probes) due to the localized surface plasmon resonance (LSPR) effect. The use of AuNPs coupled with DLS leads to excellent sensitivity with an ultralow detection limit of 0.06 pM, a marked improvement over conventional techniques. The assay can be carried out in a ‘mix-and-measure’ manner that is faster, simpler, and cheaper than the conventional methods where, in general, multiple incubation with multiple labeled reagents and repeated washing steps are required. This assay is highly specific due to the fact that the p53 REs (DNA) are conjugated onto the AuNPs, thereby suppressing any signals arising from non-binding substances and allowing drug screening in complex mediums such as cell lysate or blood serum.

The DLS equipment is a common characterization equipment used by pharmaceutical companies, providing an easy adaptation pathway for this assay to be adapted by the pharmaceutical industry to screen and validate p53 activation or reactivation drugs. In 2014, the global market value for cancer drugs reached $100 billion per annum, and is expected to increase to $147 billion by 2018. This large market promotes increased investment in cancer drug research and development evidencing an urgent need for anticancer drug screening assays targeting the p53 pathway for the development of general anticancer therapeutics.

Dynamic light scattering is a well-known analytical technique capable of analyzing particle size distribution down to the nanometer range. The particle size is determined by monitoring fluctuations in scattered light intensity caused by Brownian motion of particles in a solution. DLS has been used to measure the hydrodynamic radius or size of purified proteins and DNAs at very high concentrations. However, since all biomolecules scatter light to a similar extent, it is almost impossible to detect the binding interactions, especially at low concentrations in a complex biological sample. Therefore, the drug screening nanoplatform in accordance with the present embodiment includes a DLS probe designed with to include a gold nanoparticle (AuNP) with high scattering dimensions to enhance the DLS signal and at the same time suppress any background noise, conjugate/link with one p53 response element (RE) to allow sequence specific binding, and provide a passivated probe surface with oligonucleotides to prevent non-specific adsorption of irrelevant biomolecules.

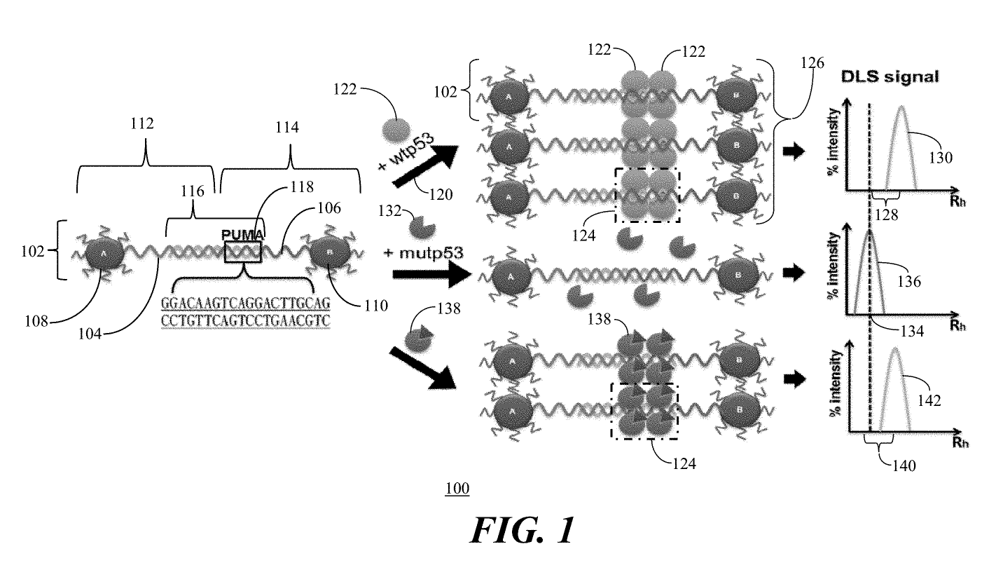

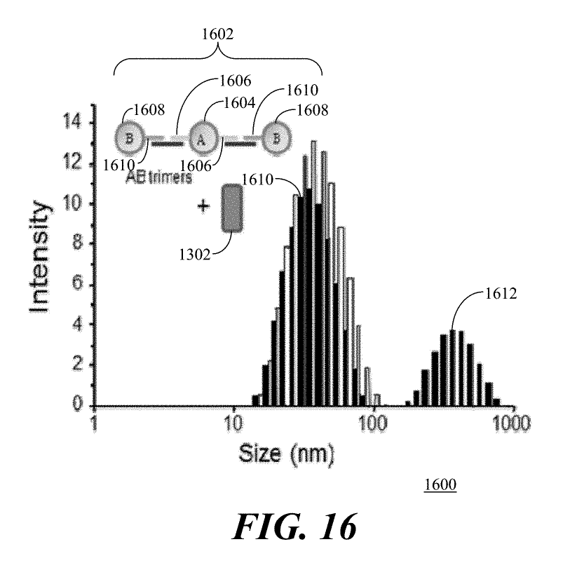

Referring to FIG. 1, a schematic illustration 100 depicts the drug screening assay principles for mutant p53 reactivation using specially designed dumbbell-shaped p53 RE-linked AuNP probes 102 as ultrasensitive sensing probes in DLS measurements in accordance with the present embodiment. While the transcription factor p53 is utilized in some embodiments discussed herein, those skilled in the art realize that the methods, systems and devices in accordance with the present embodiment can determine transcription factors interaction of many different transcription factors such as p53, p73, NF-kB, FoxP3 and the family of transcription factors referred to as signal transducers and activators of transcription (STAT) family. In addition, while the nanoparticle dumbbell probes are described as including gold nanoparticles, other plasmonic metal nanoparticles can be used in accordance with the present embodiment such as silver nanoparticles, gold nanorods or gold-silver alloy nanoparticles. Further, while two nanoparticle nanoprobes are described herein, as discussed later in regards to FIG. 16, nanoprobes in accordance with the present embodiment can include three or more nanoparticles.

Utilizing the above-mentioned criteria, the p53 RE linked AuNP (RE-AuNP) DLS probe 102 is prepared. Briefly, two 5′-thiolated single-stranded DNAs (ssDNAs) 104, 106 hereby termed A 104 and B 106, are conjugated onto AuNPs 108, 110 to form AuNP-A 112 and AuNP-B 114, respectively. After passivation with polyT oligonucleotides, AuNP-A 112 and AuNP-B 114 are hybridized to a target ssDNA AB 116 which is complementary in sequence to both probes. A p53 reaction element (RE) 118 (e.g. a PUMA sequence where p53 unregulated modulator of apoptosis (PUMA) is a pro-apoptotic protein, a member of the Bcl-2 protein family) is on the side with the AuNP-B 114. The RE-AuNPs 108, 110 are linked by one single strand of double-stranded DNA (dsDNA) between the two AuNPs 108, 110, looking like a dumbbell. The nano-dumbbell AuNP probes 102 exhibit a distinct size that is about two times larger than individual AuNPs 108, 110 as measured by DLS.

When the nano-dumbbell AuNP probe 102 is incubated 120 with wildtype p53 (wtp53) proteins 122, the proteins 122 will bind to the p53 RE 102 as a multimer 124 such as a tetramer, leading to assembling 126 or stacking of multiple nano-dumbbell probes 102. The resulting increase in hydrodynamic radius of the assembly of nano-dumbbell AuNPs probes 126 gives a significant change in a position 128 of the DLS signal 130. A similar aggregation phenomenon was observed previously with RE conjugated microbeads (1 μm) via fluorescence imaging.

In contrast, mutant p53 (mutp53) proteins 132 are unable to bind to the nano-dumbbell probes 102 and, thus, there is no change in a position 134 of the DLS signal 136 since this assay only detects proteins that specifically bind to nano-dumbbell probes 102 but receives no interference from the solution background. When a drug successfully reactivates mutp53 to restore its RE binding ability, the drug-reactivated p53 proteins will bind to the nano-dumbbell probes 102 and the extent of reactivation (i.e., effectiveness of the anticancer drug) can be evaluated by measuring a position 140 of the resultant DLS signal 142. Based on the novel sensing principle in accordance with the present embodiment, a versatile DLS-based AuNP probe assay has been designed that can advantageously be applied to quantitatively measure sequence-specific p53-DNA binding with high sensitivity and selectivity, allow high-throughput screening of p53 activation or mutant reactivation drugs in cell lysates, and evaluate the binding affinities of various DNA promoter sequences to wildtype p53.

### Application 1

Fast and Quantitative Measurement of p53 Protein-DNA Binding with High Sensitivity and Specificity in Real Time.

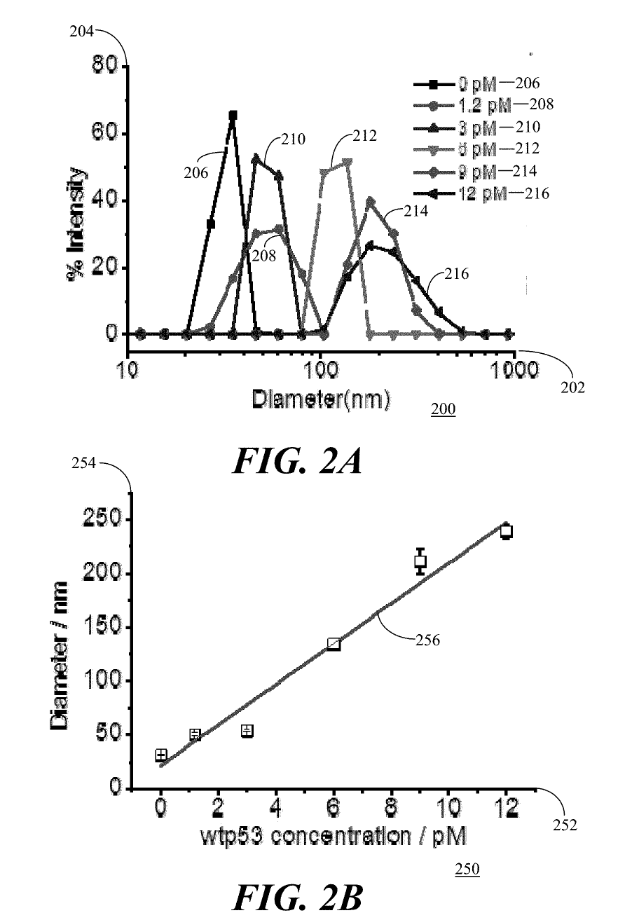

FIG. 2, comprising FIGS. 2A and 2B, depicts graphs 200, 250 of size distribution and calibration in accordance with the present embodiment. Referring to the graph 200, particle size is logarithmically plotted along the x-axis 202 and percent intensity of the scattered light from the DLS is plotted along the y-axis 204. The concentration of p53 at 0 pM 206, 1.2 pM 208, 3 pM 210, 6 pM 212, 9 pM 214, and 12 pM 216 is plotted on the graph 200. It can be seen from the graph 200 that the DLS signal shifts to a larger diameter as the concentration of wildtype p53 protein in the assay increases. Referring to the graph 250, p53 concentration in the assay is plotted along the x-axis 252 and particle size is plotted along the y-axis 254. The linear calibration plot 256 indicates that the average hydrodynamic size of the wildtype p53 bound PUMA-AuNP complex is directly proportional to the concentration of wildtype p53 proteins in the assay.

Thus it can be seen that the assay in accordance with the present embodiment demonstrates quantitative measurement of wildtype p53 interacting with the PUMA RE sequence with excellent sensitivity and ultralow detection limit. The linear range of the wildtype p53 protein concentration is from 0 to 12 pM with an ultra-low detection limit of 0.06 pM (S/N=3) achievable in accordance with the present embodiment. Furthermore, the calibration plot 256 in the graph 250 advantageously enables determination of wildtype p53 protein concentration in an unknown sample.

### Fast Detection and Real-Time Monitoring of Wtp53-DNA Binding.

Since p53-DNA binding is a time-dependent event, the ability to real-time monitor the binding interaction in a homogeneous solution is of great importance. Surface plasmon resonance (SPR) is typically considered the standard method for real-time monitoring of protein-DNA or protein-protein binding, but SPR has limitations. For instance, SPR requires surface functionalization of a probe and then detects the binding interaction at a solid-liquid interface which may introduce steric hindrance for the binding event and also does not accurately represent the physiological situation. However, accuracy is especially critical in the case of p53-DNA binding since p53 proteins tend to aggregate upon binding. Unlike SPR, the system in accordance with the present embodiment provides a direct, single-step, real-time monitoring of wildtype p53 protein binding to its RE in a homogeneous solution, without requiring surface immobilization and advantageously having the ability to better mimic actual physiological conditions.

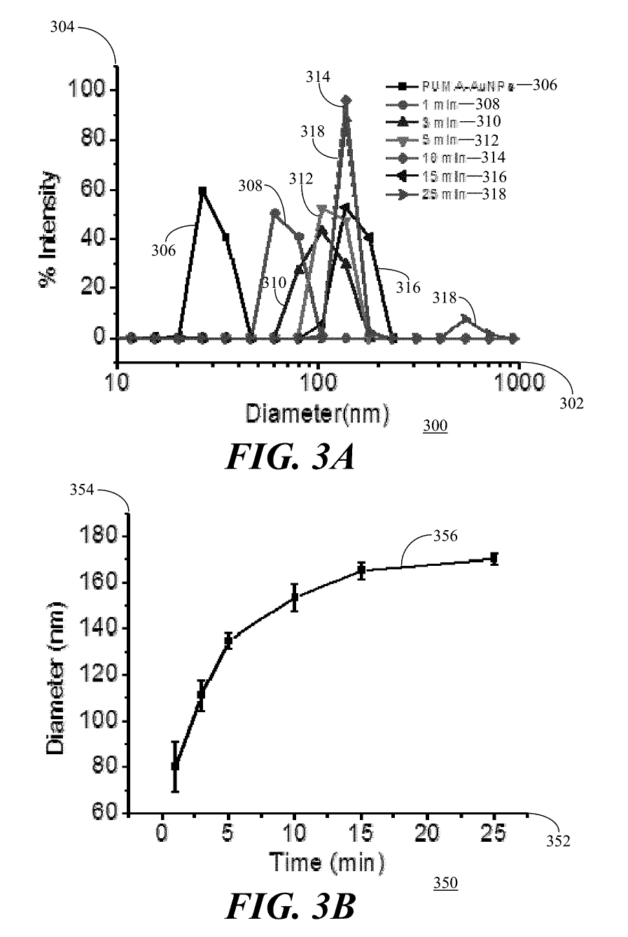

Referring to FIG. 3, graphs 300, 350 depict real time monitoring of the wildtype p53 and PUMA-AuNP binding event. The graph 300 (FIG. 3A) logarithmically plots particle size along the x-axis 302 and percent intensity of the scattered light from the DLS along the y-axis 304. The time monitored of the wildtype p53 and PUMA-AuNP binding event is shown at zero minutes 306, one minute 308, three minutes 310, five minutes 312, ten minutes 314, fifteen minutes 316 and twenty-five minutes 318 is plotted on the graph 300. It can be seen from the graph 350 that the DLS signal shifts to a larger diameter as the duration of incubation increases.

Referring to the graph 350, time is plotted on the x-axis 352 and particle size is plotted on the y-axis 354. The statistical plot 356 shows that a size shift can be observed after one minute and it increases with incubation time reaching a plateau at around fifteen minutes. The distinct size change observable after just one minute of incubation evidences that DLS promptly detects the specific binding interaction between wtp53 and RE. Thus, the present embodiment advantageously provides reduced assay time and allows faster detection.

High Sequence Specificity Wildtype p53-DNA Binding.

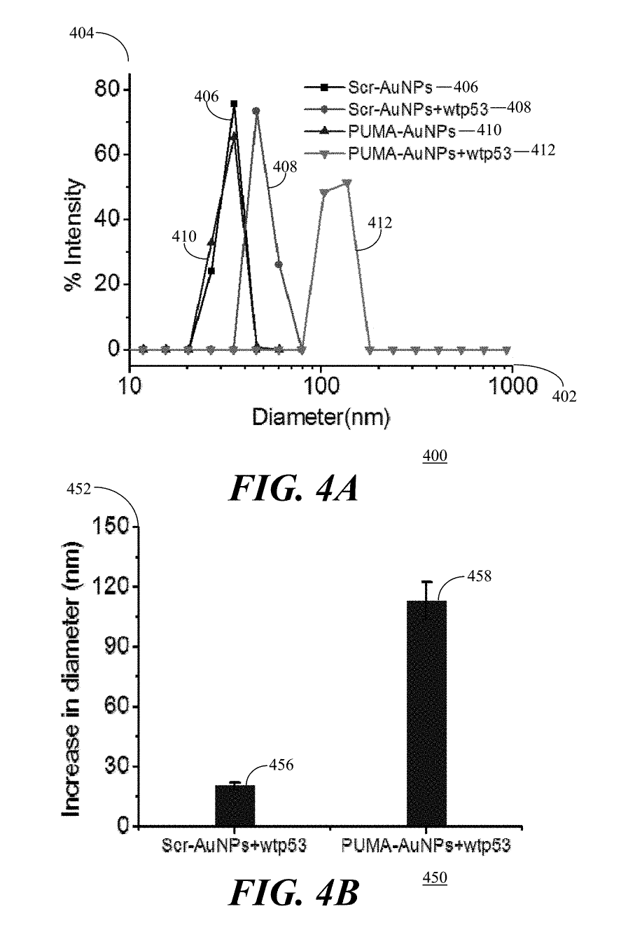

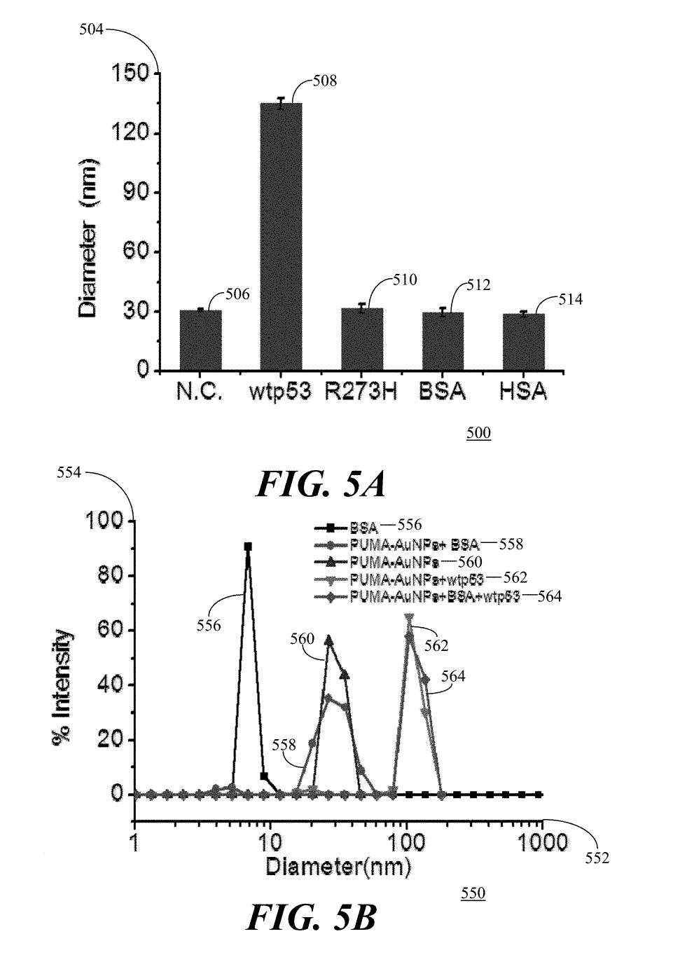

The DNA sequence selectivity in accordance with the present embodiment is demonstrated in FIG. 4 by comparing the wildtype p53 binding to the PUMA-AuNP nano-dumbbell probes containing a natural wildtype p53 binding promoter sequence to scrambled gold nanoprobes (Scr-AuNPs) linked by a scrambled DNA without a p53 binding sequence. Referring to FIG. 4A, a graph 400 depicts size distribution in a DNA sequence selectivity study where particle size is plotted along the x-axis 402 and percent intensity of the scattered light from the DLS is plotted along the y-axis 404. Both the Scr-AuNPs 406 and the PUMA-AuNPs 410 exhibited similar particle size as measured in DLS in the absence of wildtype p53 protein. Upon addition of the wildtype p53 (at approximately six pM), a significant increase in particle size was observed for the PUMA-AuNPs 412 (˜120 nm) which is approximately five-fold higher than that for Scr-AuNPs 408 (˜20 nm). This is further shown in a bar graph 550 (FIG. 5B) where the increase in diameter is plotted along the y-axis 452 where the particle size increase for the PUMA-AuNPs 458 is significantly larger than the particle size increase for the Scr-AuNPs 456. The slight increase in particles size for the mixture of wildtype p53/Scr-AuNPs 456 is due to the non-specific electrostatic interactions between negatively charged Scr-AuNPs and positively charged wtp53, showing the sensitivity of the assay in accordance with the present embodiment to differentiate both the specific and non-specific binding in addition to the superior sequence selectivity of the assay.

Excellent Differentiation of Mutant Proteins Vs. Wildtype Proteins.

The capability to differentiate wildtype p53 from mutant p53 is critical towards the success of a drug screening assay. When p53 proteins are mutated, they lose their capability of binding to specific DNA promoter sequences containing the REs. The specially designed PUMA-AuNP nano-dumbbell probe in accordance with the present embodiment easily distinguishes the binding wildtype p53 protein from the non-binding mutant p53 proteins. Referring to FIG. 5A, a bar graph 500 plots the particle size along the y-axis 504. While the wtp53-bound PUMA-AuNP probes 508 show a significant increase in size (˜100 nm) on DLS measurement, the mutant p53(R273H)/PUMA-AuNPs sample 510 was found to have the same size as that of original probe 506 (33 nm) indicating negligible binding of the mutant p53 with the PUMA-AuNPs in vitro. Similarly, other non-specific proteins such as bovine serum albumin (BSA) 512 and human serum albumin (HSA) 514 show negligible binding to the PUMA-AuNPs. Referring to FIG. 5B, a size distribution graph 550 logarithmically plots particle size along the x-axis 552 and plots scattered light intensity along the y-axis 554. More importantly, it is observed from the graph 550 that the signal from the non-specific BSA proteins 556 is almost entirely suppressed upon mixing with PUMA-AuNPs 558 as compared to the signal solely from the PUMA-AuNPs 560. The wildtype p53 562 (6 pM) binds to the nano-dumbbell probes causing an increase in particle size, and the size change remains the same in the presence of a high concentration of the non-specific proteins 564 (e.g. BSA, 4 mM). Thus, in accordance with the present embodiment, the PUMA-AuNPs can effectively suppress the background interference resulting from non-binding proteins in the detection medium (e.g., BSA as seen from the data 564 as compared to the data 562). Therefore, the PUMA-AuNP nano-dumbbell probes in accordance with the present embodiment can specifically detect binding of p53 proteins onto the RE sequences in a more complex biological medium such as cell lysate.

### Application 2

### Anticancer Drug Screening in Cell Lysates.

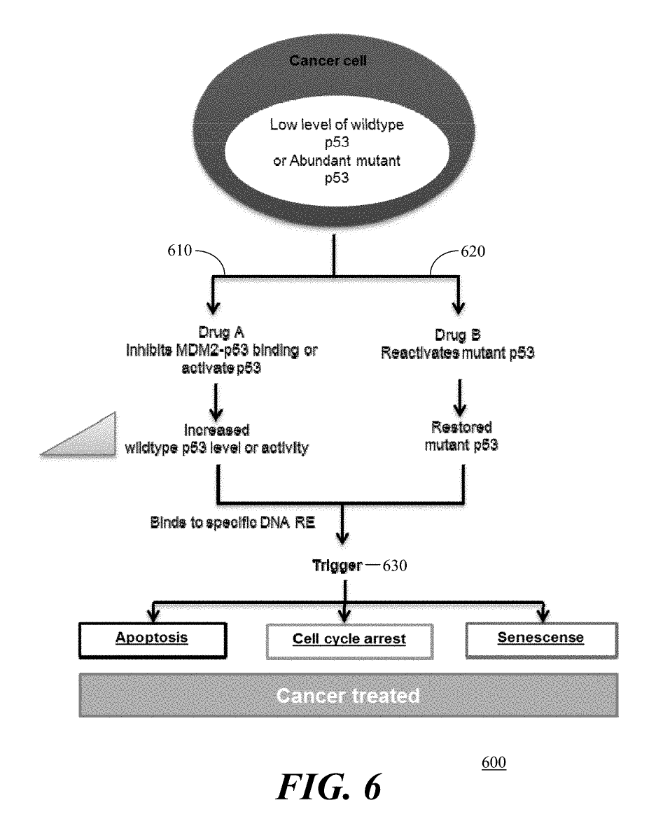

The major approaches to correct the dysfunctional p53 regulatory pathway are to inhibit the p53-MDM2 (ubiquitin protein ligase that targets p53 for degradation) interactions or to restore the functions of mutant p53. FIG. 6 depicts a summary illustration 600 of drug screening applications in accordance with the present embodiment. As depicted in the illustration 600, this technology can be used to screen for a drug/compound that increases p53 activity 610, screen for a drug/compound that reactivates mutant p53 620, and evaluate the DNA binding affinities for drug validation and verification of downstream pathways 630.

Screen for a Drug that Increases p53 Activity.

Unlike most current techniques that require purified protein samples, the assay in accordance with the present embodiment is able to directly detect the protein in cell lysates, allowing more clinically-relevant data to be obtained. Although cell lysates typically contain many other substances such as proteins and DNAs which will also scatter light, the intensity is significantly lower than that exhibited by the AuNPs due to the large scattering cross-section of AuNPs. The fact that p53 REs are conjugated onto the AuNPs probe also ensures that the detection of p53-DNA binding is highly specific and any signals arising from the non-binding substances are significantly suppressed, thereby allowing testing for native p53 protein in cell lysates.

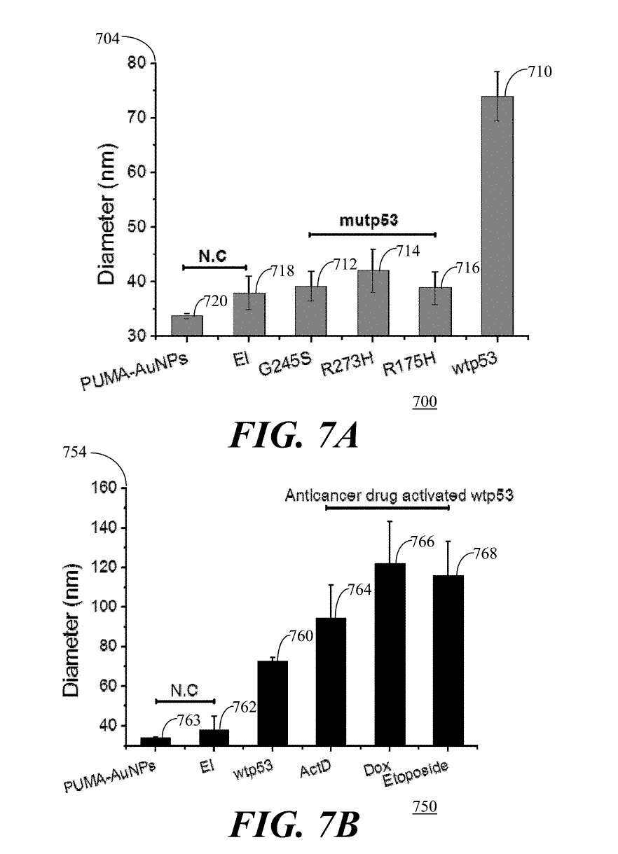

To investigate the applicability of the assay in accordance with the present embodiment to cell lysates, the H1299 Ecdysone-Inducible (EI) system for controllable and constitutive expression of p53 in H1299 cells induced by ponasterone A (PonA) was used. Referring to FIG. 7, bar graphs 700, 750 depict cell lysate analysis statistics in accordance with the present embodiment. The bar graph 700 plots size along the y-axis 704 and depicts wildtype p53 cell lysate 710 and three mutant p53 cell lysates: G245S 712, R273H 714 and R175H 716. The p53 null cell lysate (EI) 718, which serves as a negative control, gives negligible size shift from that of the PUMA-AuNP nano-dumbbell probes 720. Remarkably, clear distinctions between cell lysates with wtp53 710 and the DNA contact mutant retaining native structure (R273H) 714, the weakly stabilized mutant (G245S) 712 and the globally denatured mutant (R175H) 716 can be observed in the bar graph 700. Thus, it can be seen from the analytical results in the graph 700 that the bioassay in accordance with the present embodiment is not only sensitive and selective in the context of purified proteins but also applicable to highly complex biological samples.

Referring to FIG. 7B, the bar graph 750 plots size along the y-axis 754 and depicts detection of wildtype p53 in PonA induced p53 cell lysates 760 versus a p53 knockdown cell lysate (EI) 762 (used together with the PUMA-AuNP nano-dumbbell probes 763 as a negative control), and an anticancer drug including ActD-treated 764, Dox-treated 766, and Etoposide-treated cell lysate 768. Addition of the anticancer drugs including actinomycin D (ActD) 764, doxorubicin (DOX) 766, and etoposide 768 is expected to activate wtp53 and consequently improve the binding to the RE, where the improved binding is successfully detected as a larger diameter and is obtained for the anticancer drug treated cell lysate samples (ActD, Dox and Etoposide). The results depicted in the graph 750 demonstrate the capability of the bioassay in accordance with the present embodiment to screen for drugs that activate wildtype p53-DNA binding.

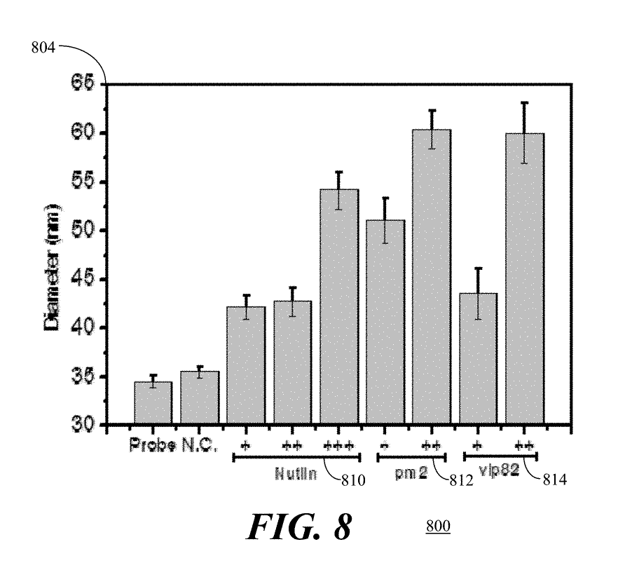

Referring to FIG. 8, detection of wtp53 expression level in drug treated HCT116 cell lysates in accordance with the present embodiment is depicted in a bar graph 800. Besides the H1299 Ecdysone-Inducible cell lines, the effect of Mdm2 inhibitors such as nutlin 810, pm2 812, and vip82 814 on HCT116 cells was investigated. These inhibitors could inhibit Mdm2 targeted wtp53 degradation so that the wtp53 protein level in the HCT116 cell lysate will increase with the concentration of the drug applied. As shown in the graph 800, the increased wtp53 level can be detected upon mixing with the PUMA-AuNP nano-dumbbell probes. However, the extent of size increase is not as high as what was observed upon anticancer drug treatment presented. One possible explanation is that PUMA-AuNP nano-dumbbell probes are sensitive to detect activated wtp53 with enhanced binding to RE, whereas the over-expression of wtp53 may not enhance DNA binding.

### High-Throughput Screening of Drugs for Mutant p53 Reactivation.

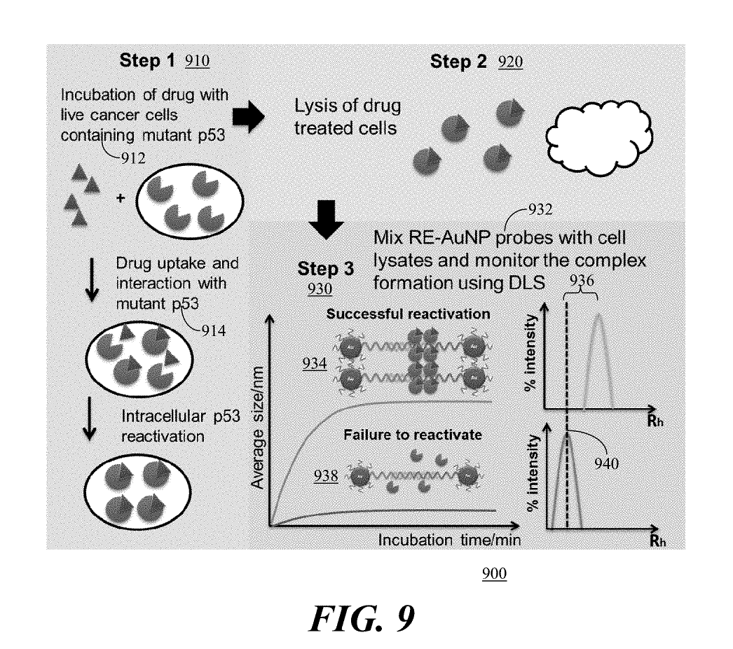

The ability to clearly distinguish wildtype p53 from mutant p53 in cell lysates provides the potential of the assay in accordance with the present embodiment for screening drugs that can effectively reactivate mutant p53 directly in cells and recovered the biological samples from cell lysates for detection. FIG. 9 depicts a schematic diagram 900 showing the process of mutant reactivation drug screening. Incubation of drug with live cancer cells expressing mutant p53 for reactivation is performed 910 (Step 1). The drug treated cells are then chemically lysed 920 (Step 2). The resultant cell lysates are next subjected 930 to incubation with p53-RE functionalized AuNP nano-dumbbell probes (Step 3). The obtained DLS readout allow evaluation of whether the drug applied has successfully reactivated mutant p53 to restore its binding affinity to RE. Failure in p53 reactivation will give rise to no change in the position of the DLS signal in accordance with the present embodiment.

Referring back to Step 1, drugs to be tested are first incubated 912 with live cancer cells expressing mutp53 proteins. Drugs are then uptaken 914 by the cells and interact with the mutp53 proteins. The drug treated cancer cells are subsequently lysed at Step 2 (920) to obtain crude cell lysates. These cell lysates are incubated with RE-AuNP probes and DLS is finally employed to evaluate the efficiency of mutp53 reactivation 932. If the reactivation is successful 934, the reactivated p53 protein will have a restored binding toward the RE, thus leading to increase in the diameter of the p53 RE-AuNP probes as measured 936 by DLS. In contrast, failure in reactivation 938 will not restore the binding of mutp53 protein to the RE sequence on the nano-dumbbell probes, thus no change in DLS signal is measured 940. Thus, the bioassay in accordance with the present embodiment is able to evaluate the extent of mutant reactivation by comparing the DLS signal obtained with the wtp53 binding to RE. The effective concentration of drugs for reactivation can also be determined by performing a series of concentration-dependent experiments during cell reactivation

There are very limited numbers of p53 reactivation drugs available currently, very possibly due to the lack of an efficient and high-throughput drug screening method. The methodology in accordance with the present embodiment provides a DLS based drug screening assay with huge potential for high-throughput screening of p53 reactivation drugs.

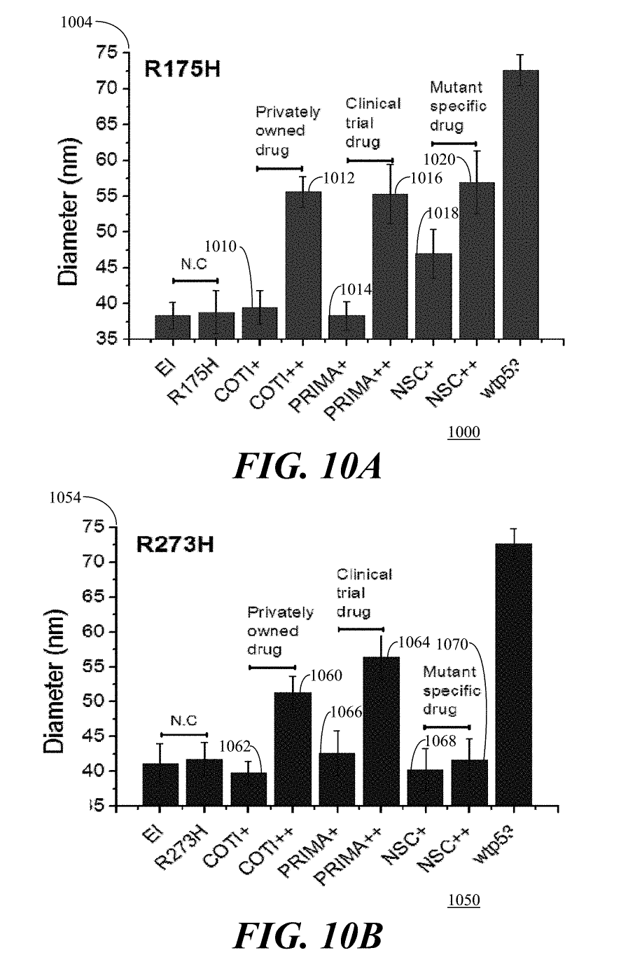

To demonstrate the feasibility and reliability of the assay in accordance with the present embodiment, three p53 reactivation drugs including COTI-2 owned by a private company, PRIMA-1^{met} undergoing clinical trial, and mutant specific NSC3198726 were tested in PonA induced H1299 cells and the results are shown in FIG. 10, comprising FIGS. 10A and 10B. The bar graphs 1000, 1050 plot particle size along the y-axis 1004, 1054 and depict screening of mutant reactivation drugs. The bar graph 1000 indicates successful reactivation of mutant p53 (R175H) to bind with PUMA-AuNP nano-dumbbell probes by COTI-2 at high concentrations (++) 1012 but not at low concentrations (+) 1010 and successful reactivation of mutant p53 (R175H) to bind with PUMA-AuNP nano-dumbbell probes by Prima-1^{met} at high concentrations (++) 1016 but not at low concentrations (+) 1014. On the other hand, the NSC drug shows effect at both low concentrations 1018 and high concentrations 1020.

Referring to the bar graph 1050, successful reactivation of mutant p53 (R273H) by COTI-2 at high concentrations (++) 1060 but not at low concentrations (+) 1062 and successful reactivation of mutant p53 (R273H) by Prima-1^{met} at high concentrations (++) 1064 but not at low concentrations (+) 1066 is shown. However, the NSC drug has no effect at either low concentrations 1068 or high concentrations 1070 due to its specificity only to R175H mutant.

Thus, in accordance with the present embodiment, higher concentrations of COTI-2 (1 μM) 1012, 1060 and Prima-1^{met} (20 μM) 1016, 1064 can successfully reactivate both p53 mutants tested (R175H and R273H) to bind with PUMA-AuNPs. In contrast, NSC reactivates R175H at both low concentrations (0.3 μM) 1018 and high concentrations (3 μM) 1020, but has no effect on the R273H mutant 1068, 1070. This is explained by the NSC's specific action on the R175H mutant. Notably, since the assay in accordance with the present embodiment is performed in a 384 multi-well plates, it is amenable for high-throughput homogeneous drug screening.

### Application 3

### Evaluation of the DNA Binding Affinities for Drug Validation and Verification of Downstream Pathways.

As aforestated, the p53 protein is a transcription factor which will bind specifically to DNA that contains RE sequences. In nature, wildtype p53 protein will bind to many promoter sequences and subsequently activate a wide range of genes for DNA repair, cell cycle arrest, apoptosis and its own degradation. As shown hereinabove, the bioassay in accordance with the present embodiment can be used for DNA selectivity study by conjugating different RE sequences to the AuNPs. However, it would be technically tedious to carry out conjugation for each DNA sequence and then screen for the binding affinity of a large number of promoter sequences. Therefore, a convenient competition assay which requires only one set of p53 RE-linked AuNPs has been designed in accordance with the present embodiment to evaluate the binding affinity of p53 protein to various promoter sequences listed in Table 1. This competition assay further allows the identification of the specific downstream pathway that is triggered upon p53 activation or reactivation, providing crucial information on the drug validation and outcome of the drug action.

TABLE 1

| | | | Gene |
|---|---|---|---|
| Name | Sequence | K_{D}/nM | Function |
| ConA | 5′- | 3.0 | Artificial |
| | GTTAGAGGGGCATGTCCGGGCATGT | | consensus |
| | CCGGGCAGA-3′ | | sequence; |
| | 3′- | | positive |
| | CAATCTCCCCGTACAGGCCCGTACA | | control |
| | GGCCCGTCT-5′ | | |
| GADD45 | 5′- | 7.7 ± 1.2 | DNA repair |
| | ATCATGAACATGTCTAAGCATGCTG | | |
| | AGCTC-3′ | | |
| | 3′- | | |
| | GAGCTCAGCATGCTTAGACATGTTC | | |
| | ATGAT-5′ | | |
| Bax a | 5′- | 73 ± 33 | Apoptosis |
| | TCATTCACAAGTTAGAGACAAGCCT | | |
| | AGCTC-3′ | | |
| | 3′- | | |
| | GAGCTAGGCTTGTCTCTAACTTGTG | | |
| | AATGA-5′ | | |
| ConAmut4 | 5′- | N.A | Mutated |
| | ACCTGGGGAATTTCCGGGAATTTCC | | ConA |
| | GCTGA-3′ | | sequence |
| | 3′- | | |
| | TCAGCGGAAATTCCCGGAAATTCCC | | |
| | CAGGT-5′ | | |
| WRNC | 5′- | N.A | Negative |
| | ATCATGAAAGGTGGATTTAGGTGGA | | control |
| | AGCTC-3′ | | |
| | 3′- | | |
| | GAGCTTCCACCTAAATCCACCTTTCA | | |
| | TGAT-5′ | | |
| Scrambled | 5′- | N.A | Negative |
| | GTTAGAGATGCGAGAGTTCAGTAAG | | control |
| | CGGGGCAGA-3′ | | |
| | 3′- | | |
| | CAATCTCTACGCTCTCAAGTCATTCG | | |
| | CCCCGTCT-5′ | | |

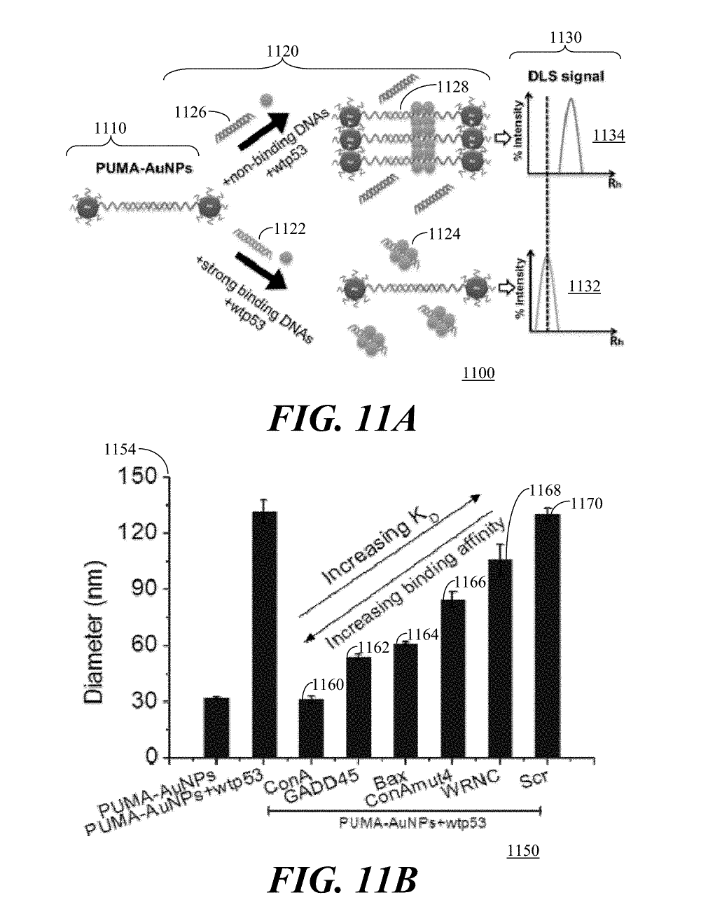

Referring to FIG. 11A, an illustration 1100 depicts evaluation of DNA sequence binding of affinity free DNA sequences in accordance with the present embodiment. First, a competition assay of free DNAs with different binding affinities for wtp53 are added to the PUMA-AuNP nano-dumbbell probes 1110 in excess, followed by the addition of wtp53 1120, and finally DLS measurement 1130. If the added free DNAs have a higher affinity to wtp53 protein than the RE-conjugated on the AuNP nano-dumbbell probes 1122, it will interact with the wtp53 preferentially 1124 and prevent the binding of wtp53 onto the AuNP nano-dumbbell probes, causing the DLS signal (i.e., particle size) to remain the same as the PUMA-AuNPs 1132. On the contrary, if the free DNA sequences have a lower binding affinity with wtp53 1126, most of the wtp53 will prefer to bind onto the RE-AuNPs 1128 than the free DNAs, leading to the increase in hydrodynamic size of the complex as indicated by the shift in the DLS signal 1134.

The dissociation constant (K_{D}) is defined as the concentration of p53 for 50% of the DNA to be bound. The lower the K_{D} value, the stronger the binding affinity between wtp53 and the tested free DNA sequence. Thus, a smaller change in the size of the complex bound probes is expected due to the competitive binding of wtp53 between the free DNAs and conjugated RE sequences on AuNPs. Referring to FIG. 11B, a bar graph 1150 plots particle size along the y-axis 1154 and shows the relative binding affinities of wtp53 towards different DNA promoter sequences as measured by our bioassay in the decreasing order as follows: ConA 1160>GADD45 1162>Bax 1164>ConAmut4 1166>WRNC 1168>Scrambled 1170. Application of this assay to the activated or reactivated p53 proteins enables determination of which promoter sequence binds strongly and consequently identifies its downstream targets (apoptosis, cell cycle arrest or DNA repair).

### Application 4

### DNA-Directed Assembly of AuNP Probe for ER (β Subtype) Detection.

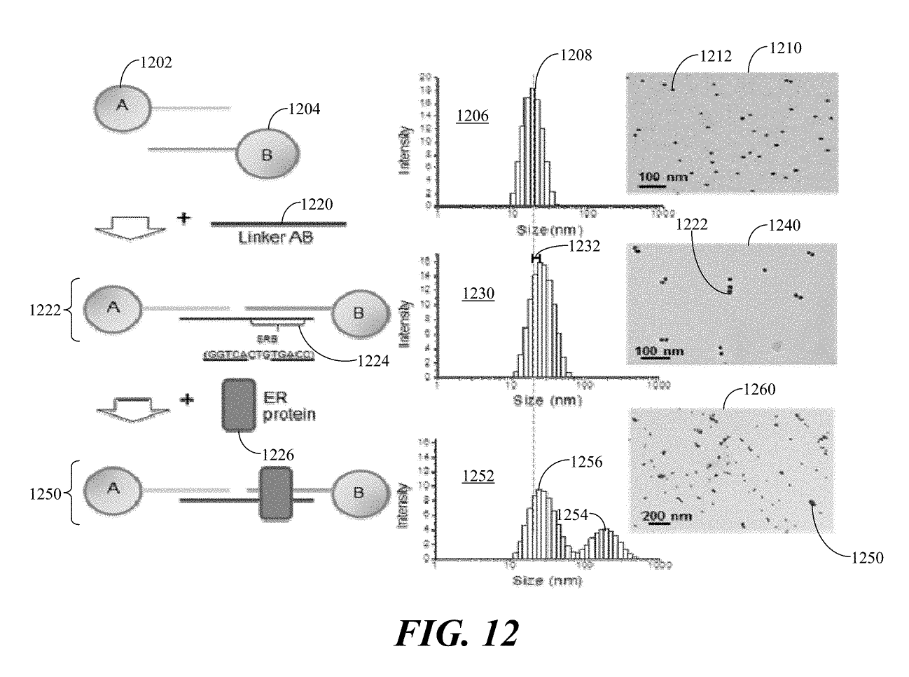

Referring to FIG. 12, an illustration 1200 depicts the design and assay principle wherein AuNPs probes of defined dumbbell probe structures linked by an estrogen response element (ERE)-containing DNA duplex are used to investigate the interactions between an estrogen receptor (ER) and their binding sites (all mentions of ER refers to the β subtype unless specifically stated otherwise). To form the AuNP probes, two sets of AuNP conjugates 1202, 1204 bearing 80 mer single ssDNA (AuNP-ssDNA) of seq A 1202 and seq B 1204 were first prepared through stoichiometric control (i.e., one DNA strand per AuNP), followed by purification using agarose gel electrophoresis. DLS measurement of these AuNP-ssDNA (seq A or B) shows in a graph 1206 a single distinct population with a size distribution peak 1208 around 20 nm, which correlates well with a TEM image 1210 of the conjugates as well-dispersed individual nanoparticles 1212.

A 100 mer DNA linker 1220 containing two 50-mer complementary sequence to seq A and seq B bridges the two sets of AuNP-ssDNA conjugates (i.e., AuNP-seq A 1202 and AuNP-seq B 1204) and forms a double-stranded DNA (dsDNA) bridged dumbbell nanostructure construct 1222. The AuNP dumbbell probe 1222 contains a consensus wildtype ERE sequence 1224 (GGTCAnnnTGACC) located at seq B 1204 where an ER 1226 can recognize and specifically bind to it. The ERE-containing AuNP dumbbell probe 1222 is then purified on agarose gel and characterized by a DLS measurement showing, in a graph 1230, a ˜10 nm rightward peak shift 1232 relative to that of the individual conjugate peak 1208 at 20 nm. The formation of the AuNP dumbbell probe construct 1222 is confirmed by a TEM image 1240. The as-formed 30 nm ERE-containing AuNP dumbbell probes 1250 can then be used as a highly specific sensing probe 1250 to detect DNA-ER binding interactions in a homogenous solution. The DLS readout in a graph 1252 shows the appearance of a ‘complex peak’ 1254 in the 200-300 nm region, which was accompanied by a decrease in the DLS signal intensity of the original dumbbell probe peak 1256 shifted to 30 nm. It is conjectured that these distinctive, two population optical signature is believed to be the result of the sequence-specific binding of ER onto the ERE-containing AuNP dumbbell probes as shown in a TEM image 1260 evidencing that the AuNP dumbbell probe nanostructures in accordance with the present embodiment can advantageously be used in conjunction with a DLS platform to study transcription factor-DNA interactions.

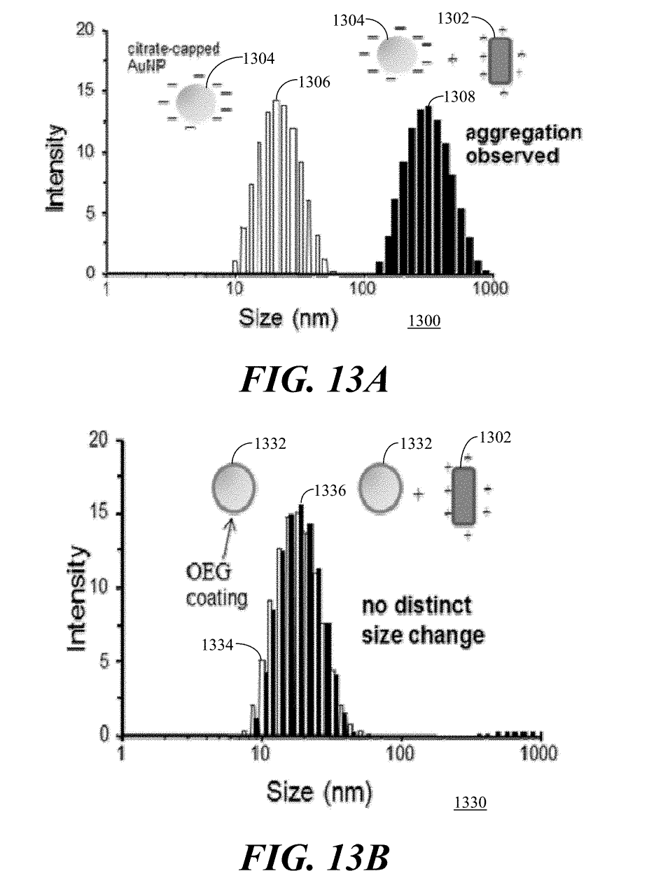

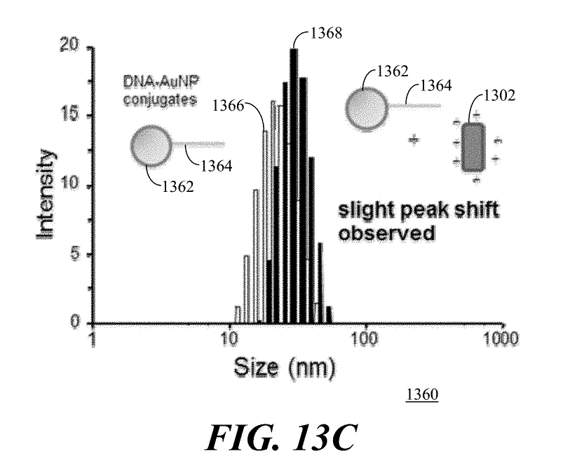

To better establish the phenomenon of ER and ERE-containing AuNP dumbbell probe interaction, DLS analysis of ER interaction with different AuNP nanostructures, namely unmodified citrate-anion capped AuNPs, OEG passivated AuNP, and AuNPs bearing one strand of ssDNA was conducted. FIG. 13, comprising FIGS. 13A, 13B and 13C, depicts graphs 1300, 1330, 1360 depicting DLS analysis of the ERβ interaction of FIG. 12 with various AuNP dumbbell probes in accordance with the present embodiment. The particle size distribution of different AuNPs systems before and after addition of 10 nM of ERβ are indicated by the ‘empty’ and ‘solid’ bar charts, respectively.

Referring to FIG. 13A, the graph 1300 depicts the DLS analysis of the ERβ 1302 interaction with unmodified citrate-anion capped AuNP dumbbell probes 1304. The citrate-anion capped AuNP dumbbell probes 1304, where an unmodified citrate-anion was used as the starting material to fabricate the DNA-linked dumbbell probes was incubated with 10 nM ER 1302. As seen from the graph 1300, this resulted in significant particle aggregation and shifted the entire population of AuNP dumbbell probes 1304 on the DLS readout from 20 nm (empty bars 1306) to around 500 nm (solid bars 1308). When the ER 1302 interacted electrostatically with AuNP probes 1304, the positive charge of the proteins 1302 negated the negative charge of the AuNP probes 1304, reducing the interparticle repulsion and inducing the irreversible, bulk aggregation of the AuNP probes 1304. It has been found that basic residues and thiol moieties on proteins can also interact with the negative charge of the AuNP probe 1304 surface.

The graph 1330 of FIG. 13B depicts the DLS analysis of the ERβ 1302 interaction with OEG passivated AuNP dumbbell probes 1332. The OEG-capped AuNP probes 1332 were formed from the citrate-capped AuNP probes 1304 (FIG. 13A) passivated with thiolated oligo-ethylene glycol (OEG) at a 500 fold OEG:AuNP ratio in an effort to investigate the effects of surface charge alterations on the system stability. As shown in the graph 1330, the OEG-capped AuNP probes 1332 were highly stable in the presence of the ER 1302, with the DLS readouts 1334, 1336 being essentially identical before and after ER addition, respectively. Thus, the AuNP probes 1332 capped with OEG retained their negative charge status and remained discrete particles and were passivated against ER 1302 and other AuNP probes 1332 and the aggregation previously observed for the citrate-capped AuNP probes 1304 in the graph 1300 was not observed.

The graph 1360 (FIG. 13C) depicts the DLS analysis of the ERβ 1302 interaction with AuNP dumbbell probes 1362 bearing one strand of ssDNA 1364. A slight rightward peak shift of ˜10 nm was observed from the DLS readouts 1366, 1368 before and after ER addition, respectively, with no complex peak further augmenting the mechanistic conjecture when the single AuNP-ssDNA conjugates 1332 were incubated with the ER 1302 described above. It is believed that the non-specific interaction of the ER 1302 with the ssDNA 1364 attached to the AuNP probes 1362 was unable to induce AuNP clustering and cause significant size increase. On the other hand, when the ERE-containing dumbbell probes were incubated with ERα, another isoform of the ER 1302, results similar to that of ERβ was observed with the appearance of a complex peak. Thus it appears that only when both the ER 1302 and ERE-containing AuNP dumbbell probes 1250 (FIG. 12) were present that a readout with the complex peak 1254 could be elicited, followed by the decrease in population of the original dumbbell probe peak. As the specific interaction between the ER 1302 and its binding site ERE is the cause for the unique optical signature of the complex peak, the complex peak 1254 provides a definitive readout for the presence of the ER transcription factor.

The ER-ERE interaction could not be studied on DLS without the transduction of the signal readout by the AuNP probes. The readouts of the ER-only, and ER-bound ERE samples (all without AuNPs) showed no significant difference from that of the buffer only. In addition, the graph 1330 depicting the DLS results of the OEG-passivated AuNPs evidences that the presence of AuNP could suppress the light-scattering signal from unbound proteins, buffers and other background noises. These factors indicate that the DLS nanoplatform in accordance with the present embodiment provides a highly sensitive and specific DLS readout with the biorecognition transduced by the unique ERE-containing AuNP dumbbell probes for the detection of target transcription factor in complicated biological samples such as blood or cell lysates that have less distinct light scattering cross sections.

### Real Time Detection and Concentration Dependence of ERE-Containing AuNP Dumbbell Probe-DLS Readout for ERβ Binding.

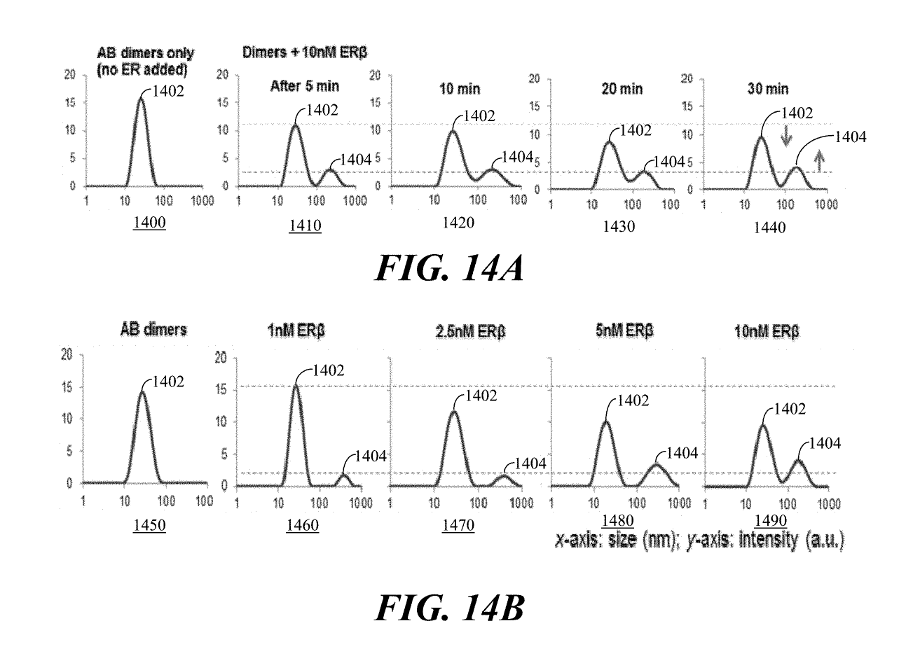

For bioassay development, it is important to quantify the amount of analytes at low detection limits, as well as to establish the rapidity of the technique. Referring to FIG. 14A, graphs 1400, 1410, 1420, 1430, 1440 depict changes in the complex peak 1404 relative to the original dumbbell probe peak 1402 over time upon adding 10 nM ER to the ERE-containing AuNP dumbbell probes. As seen in the graph 1410, the detection took only five minutes for the appearance of the complex peak 1404. Thus, the DLS nanoplatform in accordance with the present embodiment promptly pick ups and visualize any interaction between ER and ERE, mediated by the AuNP dumbbell probe probes. Over time, the size of complex peak 1404 relative to the dumbbell probe peak 1402 increased as seen in the graph 1420, 1430, 1440. This is due to the temporal effects of ER binding onto ERE-containing AuNP dumbbell probes. Such time-dependent kinetics is typical of binding processes of DNA-protein interactions.

Referring to FIG. 14B, graphs 1450, 1460, 1470, 1480, 1490 demonstrate the unique optical changes of the ‘complex’ peak 1404 versus the ‘original’ dumbbell probe peak 1402 is also ER concentration-dependent. The graphs 1450, 1460, 1470, 1480, 1490 show the particle size distribution of the ERE-containing AuNP dumbbell probes in the presence of different concentrations of ER, with larger size changes corresponding to a greater amount of ER added (all measured at thirty minutes after ER addition). The results in the graphs 1450, 1460, 1470, 1480, 1490 indicate that the amount of proteins detected can also be quantified. Thus, the sensitivity of the technique can advantageously be enhanced through further optimizations in the amount of probes used and the binding conditions used to maximize the ER binding to the ERE-containing AuNP dumbbell probes. One distinct advantage of DLS over conventional spectroscopic technique is that the amount of probes required to elicit the readout was much less as a high threshold concentration of AuNP was not required, unlike conventional spectroscopic techniques such as colorimetric or UV spectroscopy-based detection techniques, without affecting the speed and ease of readout in accordance with the present embodiment.

### Sequence and Target Selectivity of the ERβ Binding Detection System.

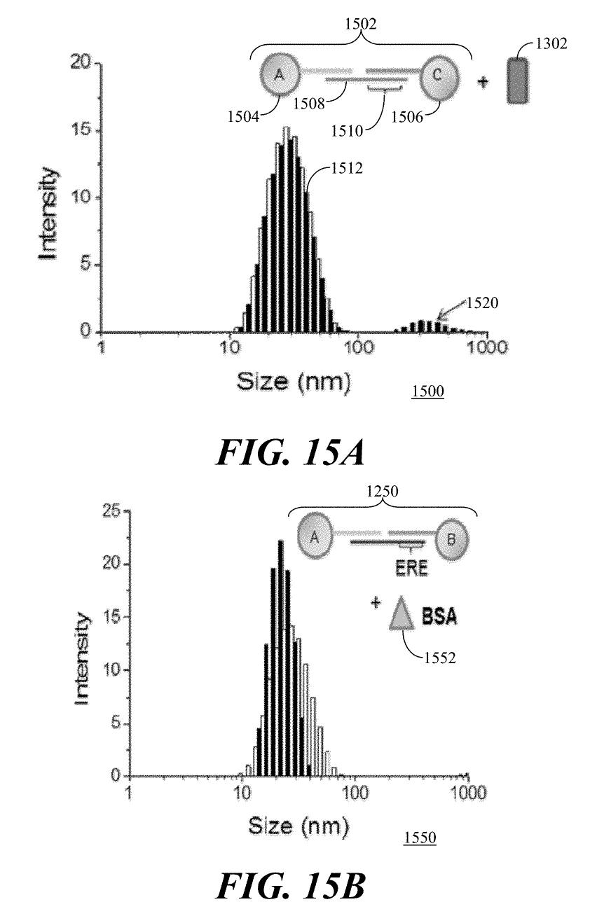

Referring to FIG. 15, graphs 1500, 1550 highlight the sequence selectivity of the present embodiment. A different type of dumbbell probe AC 1502 was fabricated from SeqA AuNP conjugates 1504 and SeqC AuNP conjugates 1506 carrying one ssDNA linked by a 100-mer AC linker 1508. Different from the ERE-containing AuNP dumbbell probes, the AC dumbbell probes contain a mutated DNA sequence 1510 located at the seq C 1506 where the core binding sequence of ERE was scrambled. When 10 nM of the ER 1302 was added to the AC dumbbell probes 1502 for DLS measurement, a complex peak 1520 was observed in the DLS readout 1512, but at a much lower intensity as compared to their ERE-containing counterparts. This appears to be attributable to the electrostatic interactions between the AC dumbbell probes 1502 and the ER 1302. However, due to the non-specific and weak binding nature of the ER-AC dumbbell probe interactions, the ensuing complex peak 1520 is at a lower intensity relative to that observed for the ERE-containing AuNP dumbbell probes.

To establish the system specificity for the target proteins, the ERE-containing AuNP dumbbell probes 1250 were queried with bovine serum albumin (BSA) 1552. At comparable concentrations of protein, the graph 1550 of the DLS readout indicates that the size of the system was essentially unchanged, and that no complex peaks were observed. Since changes in the transcription factor levels in cells are the subject of much scientific study, such as the reprogramming of stem cells and study of oncogenic pathways, any system querying the cell extract has to be minimally affected by the presence of many different proteins in a sample and not give any non-specific readouts. In accordance with the present embodiment, the graph 1550 indicates that an unrelated protein (BSA) 1552 was unable to elicit any aggregation in ERE-containing AuNP probes. While proteins are known to induce AuNP aggregation through charge interaction, the OEG passivation of the AuNP probes prevents this from happening, thereby maintaining the specificity of the DNA-bridged dumbbell probes for the ER target.

AuNPs and DLS are two highly complementary platforms as the large scattering cross section of the AuNPs facilitates a clear and distinct DLS readout. The DLS nanoplatform system in accordance with the present embodiment was designed such that ERE-containing AuNP dumbbell probes could interact with the ER 1302 through specific binding of the protein that eventually presented as a unique DLS readout wherein the localization of positively-charged ER on the ERE negated the negative charge of the AuNPs and the reduction of the electrostatic repulsion provided a driving force for their clustering. In addition, as ERβ binds to ERE as a tetramer, a few ER-bound AuNP dumbbell probes would cluster as their respective ERs are assembled or interacted non-specifically through the protein side chains. All of these factors lead to the increase in the overall size of the system, which also amplifies the intensity of the DLS readout. Typically, AuNPs aggregate when they lose their colloidal stability, which can be attributed mainly to electrostatic and steric factors, and environmental conditions as the presence of ions like Na⁺ and Cl⁻ can negate the AuNP surface charges, and such screening effects lead to increased clustering and aggregation. Generally, it is desirable to maintain the Coulombic repulsion and ensure the colloidal stability of the system until the target is introduced. The unique design with ERE localized in the dumbbell probes imparts a level of control such that only specific dumbbell probe-protein interaction can induce a change in the colloidal stability of the system, and the AuNPs will cluster to certain extent of particle stability instead of aggregating uncontrollably. This stability also ensures that readout changes, if any, must be due to the presence of the protein target. Moreover, the ER binding results in a distortion of the response element with bending towards its major groove, which inadvertently causes the AuNPs in the dumbbell probe construct to come into closer proximity. Such plasmonic coupling would also contribute to the red shift and increase in light scattering signature, which is translated as a unique DLS readout, enhancing the signal readout.

FIG. 16 depicts a graph 1600 of DLS readouts of ERE-containing AuNP trimer probes 1602 incubated with the ERβ 1302 in accordance with the present embodiment. In an effort to improve the detection process, the ERE-containing AuNP trimers 1602 consisting of a central AuNP 1604 carrying two SeqA 1606, which were bound to respective AuNP conjugates 1608 carrying SeqB 1610 were fabricated. This also created two binding sites for the ER 1302, and has the potential for more extensive interaction between higher order AuNP nanostructures and the ER 1302. It is clear from the graph 1600 that upon incubation with the ER 1302, the ERE-containing AuNP trimers 1602 showed a DLS readout with two peaks 1610, 1612, wherein the additional complex peak 1612 is once again observed. On the one hand, this validates the design of the system in accordance with the present embodiment in which AuNP nanostructures are utilized for transcription factor detection, and on the other hand, while the complex peak 1612 is located at a larger size average than that observed for the dumbbell probes, the readout was not significantly different. The presence of the added AuNP on the trimeric nanostructure 1602 could exert a steric effect and prevent the approach of the ER in binding to the ERE. Also, the trimer 1602 is not in a perfect linear form and the bridging DNA could twist and bend. As a result, the steric effect would become more pronounced, which modulates the detection outcome in spite of the electrostatic effects due to greater probe-protein interactions.

The use of nanostructures with a large scattering cross section also reduced the amount of AuNP probes required to bring about meaningful signal changes. In fact, the AuNPs samples that showed no appreciable signal on the UV-vis spectroscopy would still present a clear signal on DLS measurement, which is an additional advantage of using DLS for bimolecular detection over the conventional spectroscopic techniques. Thus, unlike conventional AuNP detection systems where the AuNP probes are used excessively, the amount of probes used here could be purposefully kept low such that even if ER is at low concentrations, their interaction with ERE could still elicit an appreciable positive readout. Further, the system in accordance with the present embodiment presents a label-free detection method where the ER is detected in its native form, as desired in biomarker sensing in general.

Thus, it can be seen that the present embodiment provides a novel ERE-containing AuNP dumbbell probe that is used for the detection of ER protein, via the signature readout on DLS. Complex peaks are observed only in the presence of ERE and ER, thus indicating both sequence specificity and protein selectivity. The quantification potential of the system has been evidenced through protein concentration dependent DLS signal outputs. Moreover, the system the system in accordance with the present embodiment can provide a DLS readout in as quickly as five minutes, thereby providing a high-throughput advantageous bioassay system for the study of not just transcription factors, but also other valuable biomarkers. The assay in accordance with the present embodiment provides a low nM level sensitivity more favorable than other AuNP-based aggregation assays that measure bulk-phase changes of particle size under UV-vis spectroscopy. In addition, the system in accordance with the present embodiment is not just limited to ER protein detection, the single-tube ‘mix and test’ AuNP dumbbell probe DLS-based bioassays in accordance with the present embodiment offer flexibility for detecting other DNA binding molecules by simply changing the conjugated DNA sequence, making the nanoprobes in accordance with the present embodiment versatile probes for use in biomedical research and diagnostic applications.

It can also be seen that the present embodiment provides a novel DLS-based anticancer drug screening assay targeting the druggable p53 pathways which include the wildtype activation and mutant reactivation. A first key aspect provided by the present embodiment is convenience. In accordance with the present embodiment, a convenient homogeneous-phase assay is provided in a single-tube format; label-free detection can be achieved with no chemical modification of the p53 proteins or the drug molecules; and a single-step “mix-and-measure” assay protocol is provided without multiple washing steps as required by conventional protocols. A second key aspect provided by the present embodiment is sensitivity and specificity. In accordance with the present embodiment, an ultralow detection limit of 0.6 pM is provided due to the strong light-scattering property of the AuNP nanoprobes. Also, the high specificity allows sequence specific detection of wildtype p53 binding using p53 RE linked AuNPs nano-dumbbell probe with negligible background interference. And reliable drug screening is provided in accordance with the present embodiment to detect drug-reactivated p53-DNA binding complexes.

A third key aspect provided by the present embodiment is efficiency. In accordance with the present embodiment, fast detection occurs within one minute. An efficient protein-DNA binding in a physiological solution is provided and the bioassay can be performed using multiwell plates with high amenability to high-throughput screening. A fourth key aspect provided by the present embodiment is cost-effectiveness. In accordance with the present embodiment, small sample volumes (1-5 μL), low p53 RE-AuNP nano-dumbbell probe concentrations (0.3 nM), and simple instrumentation (just need DLS which is available in most pharmaceutical companies) provides significant cost reduction for both drug and disease screening.

A fifth key aspect provided by the present embodiment is physiologically relevant results. In accordance with the present embodiment, the ability to test directly on cell lysates offers the following benefits: (a) provides more clinically-relevant data than using purified proteins, (b) accounts for all possible interactions between drugs and cellular molecules, (c) distinguishes differences in reactivation efficacy of various compounds, and (d) allows study of small molecule structure-activity relationships.

While exemplary embodiments have been presented in the foregoing detailed description of the invention, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of steps and method of operation described in the exemplary embodiment without departing from the scope of the invention as set forth in the appended claims.

What is claimed is:

1. A method for identifying sequence-specific transcription factor DNA interactions, the method comprising:
homogeneously mixing plasmonic metal nanoparticle probes with protein samples as an assay in multi-well plates, wherein the plasmonic metal nanoparticle probes comprise a plurality of plasmonic metal nanoparticles and a specific response element, and wherein the protein samples bind with the specific response elements to form an assembly of the plasmonic metal nanoparticle probes;
measuring particle size distribution of the assembly of the plasmonic metal nanoparticle probes in the solution by dynamic light scattering; and
determining one or more sequence-specific transcription factor DNA interactions from a curve of the particle size distribution determined from light scattered by the dynamic light scattering.

2. The method in accordance with claim 1 wherein the plasmonic metal nanoparticle probes comprise nanoparticle dumbbell probes.

3. The method in accordance with claim 1 wherein measuring the particle size distribution comprises measuring hydrodynamic radii (Rₕ) distribution of the assembly of the plasmonic metal nanoparticle probes in the solution by dynamic light scattering.

4. The method in accordance with claim 1 the protein samples bind with the specific response element in a multimeric configuration to form the assembly of the plasmonic metal nanoparticle probes.

5. The method in accordance with claim 1 wherein the protein samples comprise estrogen receptors, and wherein the specific response element comprises a estrogen response element, and wherein determining the one or more sequence-specific transcription factor DNA interactions screens the protein samples for breast cancer prognosis by determining a concentration of the estrogen receptors in the protein samples.

6. The method in accordance with claim 5 wherein determining the concentration of the estrogen receptors in the protein samples comprises determining the concentration of the estrogen receptors in the protein samples in response to the curve of the particle size distribution comprising a complex peak not found in a curve of the particle size distribution determined from the dynamic light scattering of light scattered from a sample comprising only the plasmonic metal nanoparticle probes.

7. The method in accordance with claim 1 wherein the protein samples comprise transcription factor protein samples activated or reactivated by a drug, and wherein the specific response element comprises a transcription factor-specific response element, and wherein determining the one or more sequence-specific transcription factor DNA interactions screens the drug by determining the drug's efficacy to restore transcription factor activity of the transcription factor protein samples.

8. The method in accordance with claim 7 wherein the transcription factor protein samples comprise a transcription factor selected from p53, p73, NF-kB, FoxP3 and signal transducers and activators of transcription (STAT) family.

9. The method in accordance with claim 8 wherein the protein samples comprise p53 protein samples activated or reactivated by an anti-cancer drug, and wherein the specific response element comprises a p53 specific response element, and wherein determining the one or more sequence-specific transcription factor DNA interactions screens the anti-cancer drug by determining the anti-cancer drug's efficacy to restore p53 activity.

10. The method in accordance with claim 7 wherein the assay comprises a competition assay, and wherein the competition assay further includes excess free DNA sequences containing response elements and the plasmonic metal nanoparticle probes to compete for binding with the transcription factor protein samples activated or reactivated by the drug.

11. A nanoprobe comprising:
a plurality of plasmonic metal nanoparticles; and
a DNA linker forming a link between the two plasmonic metal nanoparticles, wherein the DNA linker comprises a double stranded region encoding a specific response element.

12. The nanoprobe in accordance with claim 11 wherein the plurality of plasmonic metal nanoparticles comprises two plasmonic metal nanoparticles and the nanoprobe is a dumbbell-shaped nanoprobe.

13. The nanoprobe in accordance with claim 11 wherein the plurality of plasmonic metal nanoparticles comprise nanoparticles selected from gold nanoparticles, silver nanoparticles, gold nanorods and gold-silver alloy nanoparticles.

14. The nanoprobe in accordance with claim 11 wherein the specific response element binds with target proteins in a multimeric form.

15. The nanoprobe in accordance with claim 14 wherein the multimeric form bridges the link between the two plasmonic metal nanoparticles.

16. The nanoprobe in accordance with claim 15 wherein the specific response element comprises a specific transcription factor response element.

17. The nanoprobe in accordance with claim 16 wherein the specific transcription factor response element comprises a transcription factor selected from the group comprising p53, p73, NF-kB, FoxP3 and signal transducers and activators of transcription (STAT) family.

18. The nanoprobe in accordance with claim 17 wherein the target proteins are wildtype p53 proteins, and wherein the specific transcription factor response element comprises a specific p53 response element which binds with the wildtype p53 proteins in the multimeric form.

19. The nanoprobe in accordance with claim 18 wherein the wildtype p53 proteins comprise drug-activated wildtype p53 proteins.

20. The nanoprobe in accordance with claim 17 wherein the target proteins are mutant p53 proteins, and wherein the specific transcription factor response element comprises a specific p53 response element which binds with the drug-reactivated mutant p53 proteins in the multimeric form.

21. The nanoprobe in accordance with claim 16 wherein the specific transcription factor response element comprises a specific estrogen response element.

22. The nanoprobe in accordance with claim 21 wherein the target proteins are estrogen receptors, and wherein the specific estrogen response element binds with the estrogen receptors in the multimeric form.

23. A system for drug screening comprising:
an assay comprising a plurality of specific transcription factor DNA response elements having large light scattering dimensions when bound to drug-activated or -reactivated transcription factor proteins;
multi-well plates for combining the assay with a biological sample, the biological sample including drug-activated or -reactivated transcription factor proteins; and
a measurement device for determining binding affinity of the drug-activated or -reactivated transcription factor proteins with the plurality of specific transcription factor DNA response elements by measuring particle size dimensions in the multi-well plates by dynamic light scattering.

24. The system in accordance with claim 23 wherein the plurality of specific transcription factor DNA response elements are incubated onto a plurality of nanoprobes.

25. The system in accordance with claim 24 wherein the specific transcription factor DNA response elements comprise transcription factors selected from the group comprising p53, p73, NF-kB, FoxP3 and signal transducers and activators of transcription (STAT) family.

26. The system in accordance with claim 25 wherein the transcription factors comprise p53 transcription factors, and wherein the system provides a p53 pathway system for anti-cancer drug screening.

27. The system in accordance with claim 24 wherein the plurality of nanoprobes comprise a plurality of plasmonic metal nanoparticles selected from gold nanoparticles, silver nanoparticles, gold nanorods and gold-silver alloy nanoparticles.

28. The system in accordance with claim 27 wherein the plurality of plasmonic metal nanoparticles comprise two plasmonic metal nanoparticles, and wherein the plurality of nanoprobes comprise a plurality of dumbbell-shaped nanoprobes.

29. The system in accordance with claim 28 wherein each of the dumbbell-shaped nanoprobes comprises the pair of plasmonic metal nanoparticles connected by a DNA linker comprising a double-stranded region encoding one of the plurality of specific transcription factor DNA response elements.

30. The system in accordance with claim 23 wherein the biological sample comprises a purified protein sample.

31. The system in accordance with claim 23 wherein the biological sample comprises a cell lysate.

32. The system in accordance with claim 31 wherein the cell lysate comprises a crude cell lysate.

33. The system in accordance with claim 23 wherein the measurement device determines binding affinity of the drug-activated or -reactivated transcription factor proteins with the plurality of specific transcription factor DNA response elements by measuring hydrodynamic radii of bound particles in the multi-well plates by dynamic light scattering.

34. The system in accordance with claim 23 wherein the assay further includes excess DNA sequences containing response elements and/or transcription factor protein samples to compete with the drug-activated or -reactivated specific transcription factor protein samples for binding with the specific transcription factor DNA response elements.

35. A system for disease screening comprising:
an assay comprising a plurality of specific DNA response elements having large light scattering dimensions when bound to receptor elements;
multi-well plates for combining the assay with a disease screening biological sample; and
a measurement device for determining binding of the specific DNA response elements with the receptor elements in the disease screening biological sample by measuring particle size dimensions in the multi-well plates by dynamic light scattering.

36. The system in accordance with claim 35 wherein the plurality of specific DNA response elements comprise a plurality of estrogen specific DNA response elements, and wherein the receptor elements comprise estrogen receptors, and wherein the disease screening biological sample comprises a breast cancer screening biological sample, the measurement device screening for breast cancer by determining binding of the estrogen specific DNA response elements with the estrogen receptors in the breast cancer screening biological sample by measuring particle size dimensions in the multi-well plates by dynamic light scattering.

37. The system in accordance with claim 36 wherein the specific DNA response elements are encoded onto gold nanoparticle nano-dumbbell probes.

38. The system in accordance with claim 37 wherein the gold nanoparticle nano-dumbbell probes comprise gold nanoparticles.

39. The system in accordance with claim 38 wherein each of the gold nanoparticle nano-dumbbell probes comprises a pair of gold nanoparticles linked by a strand of DNA, wherein the DNA linker comprises a double-stranded region encoding the estrogen specific DNA response elements.

40. The system in accordance with claim 36 wherein the measurement device determines the binding of the estrogen specific DNA response elements with the estrogen receptors in the breast cancer screening biological sample by measuring hydrodynamic radii of bound particles in the multi-well plates by dynamic light scattering.

41. The system in accordance with claim 40 wherein the measurement device determines the binding of the estrogen specific DNA response elements with the estrogen receptors in the breast cancer screening biological sample by determining presence of a complex peak in a curve of the hydrodynamic radii distribution of the bound particles in the multi-well plates not found in a curve of hydrodynamic radii distribution determined from dynamic light scattering from a sample comprising only the plurality of estrogen specific DNA response elements.
